(19) United States
(12) Patent Application Publication
(10) Pub. No.: US 2024/0057929 A1
(43) Pub. Date: Feb. 22, 2024

(54) SYSTEM AND METHOD FOR EVALUATING STIMULATION OF TISSUE

(71) Applicant: Medtronic Xomed, LLC, Jacksonville, FL (US)

(72) Inventors: Cristina G. Schuetz, Eden Prairie, MN (US); Bhrathi Kasaba Venkatagiri, Plymouth, MN (US); John Ryan Shore, Jacksonville, FL (US); Christopher L. Fair, Jacksonville Beach, FL (US)

(21) Appl. No.: 18/352,383

(22) Filed: Jul. 14, 2023

Related U.S. Application Data
(60) Provisional application No. 63/398,240, filed on Aug. 16, 2022.

(51) Int. Cl. A61B 5/397; A61B 5/00

(52) U.S. Cl. CPC A61B 5/397; A61B 5/7264; A61B 5/74

(57) ABSTRACT

A monitoring system may include a processor and display system for displaying results from the monitoring. A user may be in a sterile field away from the processor and display system and selected input devices. The processor may execute instructions to analyze a selected signal and, if selected, display the analysis.

### CROSS-REFERENCE TO RELATED APPLICATIONS

This application claims the benefit of U.S. Provisional Application No. 63/398,240, filed on Aug. 16, 2022. The entire disclosure of the above application is incorporated herein by reference.

### FIELD

The present disclosure relates to stimulation, and particularly to nerve stimulation and evaluating related signals.

### BACKGROUND

The background description provided herein is for the purpose of generally presenting the context of the disclosure. Work of the presently named inventors, to the extent the work is described in this background section, as well as aspects of the description that may not otherwise qualify as prior art at the time of filing, are neither expressly nor impliedly admitted as prior art against the present disclosure.

A nerve of a patient may be stimulated by applying electrical energy to the nerve via a stimulation probe. The stimulation probe may include a stimulating electrode tip. A procedure user, such as a surgeon, may touch a location on a patient with the electrode tip to provide a voltage and/or current to a location on the patient and stimulate nerve activity and may result in a muscle response (e.g., muscle activity). A return (which may be an anodal) needle may be attached; such as via a wire, to the mono-polar stimulation probe; and to the patient away from (i) sensors, and (ii) an area being stimulated. The sensors can include electrodes that are attached to the patient and used to monitor the muscle activity.

Nerve monitoring systems such as the NIM-Response® 3.0 and/or NIM-Neuro® 3.0 nerve monitoring systems, sold by Medtronic, Inc., may be used to monitor electromyography (EMG) responses. Monitoring the EMG responses may be used to determine whether one or more nerves has been damaged during a select procedure. In various systems, the monitoring system may be controlled by a monitor user that is spaced apart from a procedure user and a subject. Therefore, the monitor user may require instructions from the procedure user to operate the monitoring system according to a selected use by the procedure user.

### SUMMARY

A stimulation probe is provided and includes at least a first electrode. The stimulation probe may further include a second electrode, particularly if a bipolar stimulation probe is selected for use. The stimulation probe system may further include at least one of a control module and/or switches. In various embodiments, the switches may transmit a signal to the control module to increase a stimulation signal, decrease a stimulation signal, record a signal at a selected time, and/or provide control signals to the stimulation system. A user may use the evoked potential nerve monitor system for Intermittent monitoring and nerve locating and assessment with a stimulation probe. “Continuous Intraoperative Nerve Monitoring” (CIONM) may be performed to automatically assess nerve health by Automatic Periodic Stimulation (APS) stimulation electrode.

The stimulation probe may be a part of a nerve monitoring system that may be used to monitor the integrity of a nerve. Nerve monitoring systems may include hardware such as that include with the NIM-Response® 3.0, NIM-Neuro® 3.0, and/or NIM Vital® nerve monitoring systems, sold by Medtronic, Inc., that may be used to monitor electromyography (EMG) responses. The stimulation probe, however, need not be used with a nerve monitoring system. The EMG may an evoked EMG that is sensed due to the introduction of a stimulation to the nerve of the subject. The evoked EMG, however, may be sensed and determined as a “true” evoked EMG as discussed herein. The evoked EMG is generally an electromyography response of the muscle (e.g., Compound Muscle Action Potential (CMAP) or Motor Unit Potential (MUP), Motor Evoked Potential (MEP), Motor Unit Action Potential (MUAP)) that is sensed with a sensor, as discussed herein.

The monitoring system may include a processor, a memory, and/or a display system for displaying results from the nerve monitoring. The subject may be monitored with the monitoring system for a selected procedure. The procedure user may include a surgeon. The surgeon may be sterile for the selected procedure. Switches may be connected to a monitoring and/or stimulating instrument during a procedure. Switches may also or alternatively be connected adjacent to or to the surgeon and connected with the instrument and the monitoring system. The switches may be sterile and appropriate for placement in the sterile field.

The monitoring system may further include instructions that may be executed with the processor to identify a selected signal. As noted above, the monitoring system may include at least one receiving electrode that may receive a signal. The signal may be based on a stimulation provided with the monitoring system. The signal, however, may include noise due to other electrical impulses that are received at the receiving electrode. The monitoring system may receive and analyze the signal to identify those that are relevant EMG signals (e.g., evoked EMG responses) and ignore and/or identify those that are not relevant signals, including EMG responses that are note evoked EMG responses.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 is a schematic environmental view of a stimulation instrument used during a procedure;

FIG. 2 is an illustration of waveforms of signals, according to various embodiments;

FIG. 3 is a list of attributes of a signal, according to various embodiments; and

FIG. 4 is a flowchart of an analysis of a signal, according to various embodiments.

Corresponding reference numerals indicate corresponding parts throughout the several views of the drawings.

### DETAILED DESCRIPTION

Example embodiments will now be described more fully with reference to the accompanying drawings.

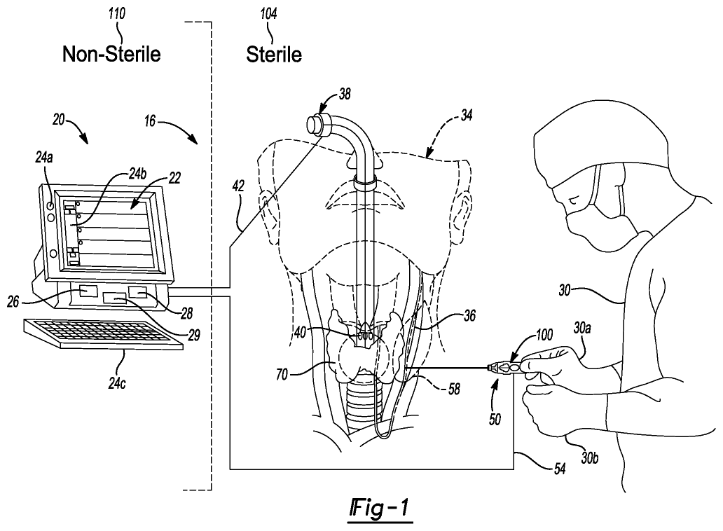

With initial reference to FIG. 1, a monitoring system 16, such as a NIM® and/or NIM Vital® nerve integrity monitoring system, sold by Medtronic, Inc. having a place of business in Minneapolis, MN, is illustrated in an environmental setting. The monitoring system 16 may also include portions similar to those described in U.S. Pat. No. 9,955,882, issued May 1, 2018 and U.S. Pat. No. 10,849,517, issued Dec. 1, 2020, both incorporated herein by reference. The monitoring system 16 may include a monitor assembly 20 that has a display screen or device 22 and one or more input devices. The input device may include one or more systems or structures to input commands of information such as a button and/or knob 24a, a touch screen 24b, a keyboard 24c, or other appropriate input devices. Input devices may also include other tactile input devices, audio input devices, visual input devices, etc.

The monitor assembly 20 may further include a processor 26 and a memory 28. It is understood that the processor 26 may access the memory 28 to execute instructions stored thereon or access other data stored with the memory 28. The memory 28 may include a physical memory, such as a spinning hard disk drive, solid state memory, or other appropriate types of memory. Further, the memory 28 may not be incorporated into the monitor assembly 20, but may be accessed by processor 26, such as via a communications network. The processor 26 may be a general purpose processor that is operable to execute instructions for generating a selected output, as discussed further herein. The processor 26 may further include onboard memory. Moreover, the processor 26 may include a specific purpose processor such as an application specific integrated circuit (ASIC). In various embodiments, however, the processor 26 may execute instructions stored on memory 28, which may be a non-transitory memory, to provide an output. The output may be an appropriate output such as an auditory output (e.g., with a speaker which may be associated with the monitor assembly 20), visual output for display on the display device 22, tactile output, etc. The processor 26 may, however, access and execute instructions that can be stored on a memory separated from the memory 28.

The monitoring system 20 may further include a stimulation portion and/or generator 29. The stimulation portion may be configured to generate a voltage based upon control (e.g., a signal) by the processor 26. The processor 26 may execute instructions of a program stored on the memory 28 and/or control by a user 30. As discussed herein, the monitoring system 20, therefore, may be operated to generate a stimulation at or with a stimulation instrument, as discussed herein, based upon control of the user 30.

The information displayed on the display device 22 may include information selected by the user 30. The selection made by the user 30 may be desired or selected information regarding a subject 34. The subject 34 is illustrated as a human subject, but it is understood that the subject may be any appropriate living subject, including non-human subjects. Further, the monitoring system 16 may be used with non-living subjects. Non-living subjects may have systems that are selected to be monitored for selected activity, such as electrical activity, training activities, and the monitoring system 16 may be used. In selected embodiments, however, the user 30 may be performing a surgical procedure on the subject 34. The user 30, therefore, may select to monitor nerve response and/or integrity such as by monitoring electromyography (EMG) responses. For example, EMG responses that are induced by the monitoring system 16 in the subject 34. An EMG response that is sensed with the monitoring system 16 following an inducement with the system 16 may be referred to as an evoked EMG or evoked EMG response. The signal related to the evoked EMG may be an evoked signal or evoked EMG signal.

One or more stimulation or monitoring assemblies may be incorporated in the monitoring system 16 and connected with the monitor assembly 20. For example, in various procedures on a thyroid 70, such as a thyroidectomy or other thyroid surgeries, monitoring of a recurrent laryngeal nerve (RLN), a vagus nerve, or other appropriate nerve 36, in the subject 30 may occur. The procedure may occur via an incision 58. Other or alternative nerves may also be monitored, including other selected cranial (e.g., Intracranial, Extracranial, Peripheral) nerves and/or spinal nerves. Monitoring of the RLN may include a nerve monitoring esophageal tube 38, which may have one or more conductive electrodes 40 that are in contact with selected portions of the subject 34. The electrode 40 may be affixed to an exterior of the tube 38 and/or incorporated into the structure of the tube 38. The electrode 40 can be connected to the monitor assembly 20, via a connection 42. In various embodiments, however, the connection 42 may be a wired and/or a wireless connection 42 between the monitoring assembly 20 and the electrode 40. In various embodiments, the monitoring assembly 20 may be positioned in a non-sterile region 110 while the subject 34 and the user 30 are in a sterile region 104. Thus, signals may be transmitted between the two regions 110, 104.

In addition, other instruments may be connected to the monitor assembly 20, such as electrode assemblies, including an electrode that may send or receive periodic stimulation pulses. The received pulses may be received automatically and/or generated automatically by the system 16. As noted above, a stimulation instrument may be used with the system 16, in various embodiments, one or more stimulation instruments 50 may be used. The stimulation instrument 50 may be connected to the monitor assembly 20 with a connector 54. The connector 54 may allow for a physical connection between the stimulation instrument 50 and the monitoring assembly 20. The connector 54 may include a conductive member (e.g., a metal wire, conductive polymer, etc.). The stimulation instrument 50 may include various instruments such as surgical instruments and the like. Examples of various types of stimulation instruments include those disclosed in U.S. Pat. No. 10,039,915 issued on Aug. 7, 2018 and U.S. Pat. App. Pub. No. 2016/0287112 published on filed Oct. 6, 2016; both incorporated herein by reference. In various embodiments, however, the connection may be a wireless connection between the monitoring assembly 20 and the stimulation instrument 50.

According to various embodiments, the instrument 50 may include the switches 100. It is understood that in various embodiments the instrument 50 may be provided to include features either separately and/or in combination as discussed further herein. The switches 100 may be operated by the user 30, such as with a first hand 30a and/or a second hand 30b. The switches 100 may include one or more buttons that may be used to increase or decrease a simulation. For example, the switch 100 may be operated to increase a stimulation and/or decrease stimulation. The switch 100 may be used to transmit a signal to the monitoring assembly 20, via a connection 54. The connection 54 may be a physical connection (e.g., wired) and/or a wireless connection.

The stimulation instrument 50 may be positioned by the user 30 to stimulate the nerve 36 at any appropriate time. Additionally, it is understood that various other electrodes may be provided to stimulate the nerve 36. Therefore, the simulation instrument 50 is merely exemplary. Regardless, the monitoring system 16 may be provided to stimulate under 36 and an evoked response may be sensed by the sensing electrode, including electrode 40. It is further understood, however, that sensing electrodes may be positioned in an appropriate location on the subject 34 such as on an arm, leg, trunk, or other appropriate area. The sensors 40 on the tube 38 are merely exemplary.

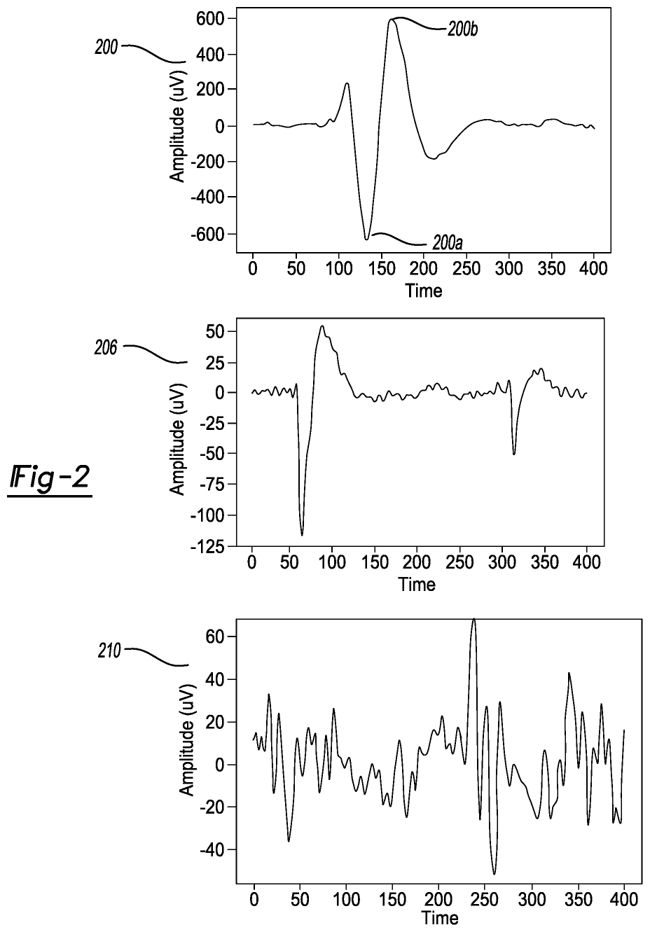

Regardless, the sensors may be used to sense an evoked response in the muscle due to stimulation of the nerve 36. The monitoring system 20 may receive a signal from the sensors 40 due to the stimulation provided by the stimulation instrument 50. The signal that is an evoked response in the muscle may be sensed and/or determined by the monitoring system 20, as discussed further herein. The sensor 40 may also receive other signals and may transmit the signal to the monitoring system 20. With continuing reference to FIG. 1 and addition reference to FIG. 2, various signals may be a positive or true EMG evoked signal 200. The true evoked EMG signal 200 may also be referred to as a positive or real evoked EMG and relates to an evoked EMG response in the subject due to the nerve 36 being stimulated with the stimulation instrument 50 or the like. A signal may be a false-positive EMG signal 206. The false-positive EMG signal may also be referred to as a not real or not true evoked EMG signal and may have some attributes similar to a true evoked EMG signal but is not due to a stimulation of the subject. A signal may also be a noise signal 210. As discussed further herein, the positive evoked EMG signal 200 may be identified with the monitoring system 20 by executing selected instructions, including an algorithm accessed thereby. The monitoring system 20, therefore, may also identify and/or exclude the false-positive signal 206 and the noise signal 210. Therefore, the user 30 may be able to better identify and understand positive evoke EMG signals and understand the condition of the nerve 36 during a procedure.

In the system a determination of the positive EMG response may include various characteristics that may be determined or understood. For example, a peak to peak threshold may be identified. A peak to peak threshold may be between an initial peak 200a and a secondary peak 200b. The threshold may be set at any appropriate threshold, such as 100 microvolts (μV) or more. The threshold may also be determined and input by the user, such as the user 30. The threshold may be used to assist in identifying a signal that is possibly an EMG signal, and may be used to define a positive evoked EMG. Other signals may be similar to an EMG, such as the false positive EMG 206. Therefore, various other attributes may be analyzed to determine whether the signal is a positive evoked EMG. Further various other signals, such as the noise signal 210 may also include characteristics similar to the positive evoked EMG 200. The various parameters may be identified and analyzed in an algorithm to assist in determining and labeling a signal as a positive evoked EMG. False positive waveforms attributes may include non-EMG Exclusion attributes, for example a Frequency outside-EMG-band, Latency out-of-range, monophasic response.

The attributes that may be associated with the signals received by the monitoring system 20 may include a plurality of attributes regarding the signal. In various embodiments, the type of procedure may be used refine or select attributes that are included in the algorithm, as discussed herein. Surgical procedure type nerve type can be used to refine True EMG (inclusion) or False Positive (execution) criteria for True EMG attributes. The attributes may include phase, amplitude, timestamp, latency, or other attributes. Selected one or more of the attributes may be incorporated into an algorithm to assist in identifying the positive evoked EMG. The algorithm may be incorporated into a program that is executed by the processor of the monitoring system 20. Therefore, when a signal is received and analyzed by the algorithm, a determination of whether or not it is a positive EMG may be made. If a positive EMG signal is determined, a notification (e.g., signal) may be provided to the user, such as an audio signal, visual signal, haptic signal, or other appropriate signal.

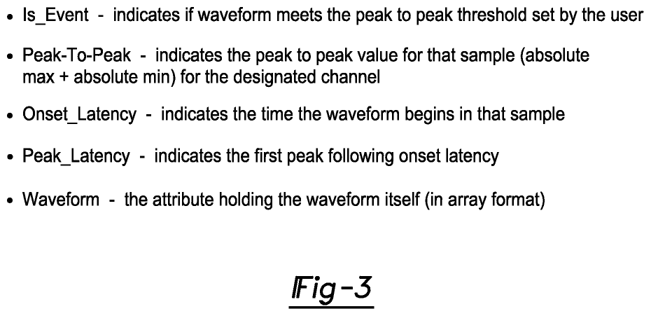

In making a determination that a signal is a true evoked EMG signal or not a true evoked EMG signal one or more attributes that may be attributed or identified in the signal that is/are received by the monitoring system 20. The attributes may include a plurality of attributes. The attributes may also be referred to herein as parameters. Possible attributes may include more than 200 attributes. Nevertheless, selected attributes have been determined to assistant in efficiently identifying positive evoked EMG signals, as illustrated in FIG. 3. The selected attributes may include a selected number of attributes, including one or more attributes including one or more of the following. A first attribute may be referred to as Is_Event and includes that the signal meets the selected peak to peak threshold (which may be set by the user), as discussed above. A second attribute may be referred to as a Peak-to-Peak value which may include an absolute maximum and an absolute minimum for each of the peaks in the signal. The absolute maximum value may be a selected value such as 10,000 microvolts (μV) and the absolute minimum value may be 0 μV. The absolute maximum may, however, be selected to be about 1,000 μV to about 100,000 μV. Also, the absolute minimum value may be about 0 μV to about 10 μV. A third attribute may be referred to as Onset_Latency, which may depend on surgical and/or nerve type, and includes a time when the wave form signal begins in the sample. The timing for the beginning of the wave form may be about 3 milliseconds, including about 1.5 milliseconds to about 6 milliseconds including the time for a signal portion (e.g., above a selected baseline) to appear after latency. A fourth attribute may be referred to as Peak_Latency and includes a time for the wave form signal to reach a maximum amplitude, including the time for a first peak to appear after latency. An exemplary time for the wave form to reach its peak amplitude may be about 4.5 milliseconds, including about 4 milliseconds to about 5 milliseconds. A fifth attribute may be referred to as a Waveform and is the wave form itself which may be identified or characterized in various formats, such as in an array format. The array format may include information which may include a shape and such as the amplitude of signal, biphasic shape, polyphasic shape or duration.

The monitoring system 20 including the memory system 28 and/or the processor system 26 is operable to access a set of instructions that may be a computer program in the memory for identifying the positive EMG signals. The computer program may include an algorithm that includes instructions to analyze the signal that is received by the monitoring system 20. In addition or alternatively thereto, the monitoring system 20 may include a machine learning system that is trained to identify the true or positive EMG signal. The monitoring system 20 may analyze the signal and provided outputs to the user 30 when the signal is identified. As noted above, various attributes may be used to identify the signal as a true or positive evoked EMG signal 200.

According to various embodiments, the algorithm may include the various attributes that are provided of equal weight. In various embodiments, the attributes may be weighted individually in an algorithm. Weights could be determined dynamically, configurable, or adjustable. The algorithm may then identify the signal that is received as a true or positive evoked EMG and/or identify the signal as not a true evoked EMG signal. An appropriate notification may then be made to the user 30.

In the various embodiments, the system to analyze of the incoming signal and determine whether the signal is a true or positive evoked EMG and/or not a true evoked EMG may be one or more machine learning systems. The machine learning systems may incorporate one or more machine learning system or algorithms such as a Supported Vector Machine, gradient boost, and XGBoost (such as provided by NVIDIA having a place of business at Santa Clara California.) The machine learning system may also be a customized machine learning system, such as a customized version of those noted above. Further, the machine learning system may be fully custom to the determination of the signal and include various features such as Artificial Intelligence.

Whether an algorithm or a machine learning system, the monitoring system 20 is provided to analyze the incoming signal and provide a determination or characterization of the signal for the user 30. The characterization may include identification of the signal as a true or positive evoked EMG or not a true a positive evoked EMG. In various embodiments, the true or positive evoked EMG may be identified on the display device 22 and/or otherwise identified to the user 30. Therefore, the user 30 may understand that a selected and determined signal is a true or positive evoked EMG signal sensed by the system 20.

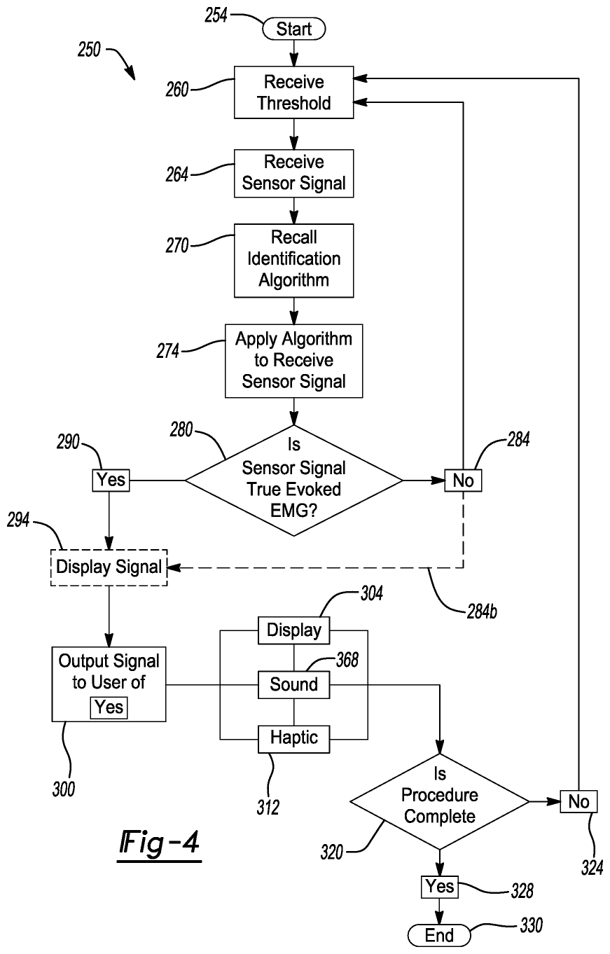

The monitoring system 20, including the processor 26, may execute instructions including the algorithm to identify the signal according to a selected method 250 as illustrated in FIG. 4. The method 250 may be performed by the processor 26 of the monitoring system 20 to identify the signal received by the monitoring system 20. As discussed above, the sensors 40 may sense a signal and transmit the signal to the monitoring system 20. The signal may be identified as a true or positive evoked EMG which may be based upon the stimulation from the stimulation instrument 50.

The method that 250 may begin at start Block 254. After the start Block 254 a threshold may be received in Block 260. The threshold received in Block 260 may include a threshold identified or selected by the user 30. A threshold may be any appropriate threshold, such as a threshold of 100 μV between respective peaks of the signal, as illustrated in FIG. 2. Any other appropriate threshold may also be identified or selected by the user 30. The threshold may assist in identifying the signal or a signal to be analyzed by the monitoring system 20, including the processor 26.

Thereafter, the monitoring system 20 may receive the sensor signal in Block 264. The received sensor signal may be received from the sensor 40 or any other appropriate sensor on the subject 34. The signal received may be analyzed by recalling the identification algorithm in Block 270. The identification algorithm may include the algorithm and/or machine learning system as discussed above. This may include the one or more attributes as noted above to be analyzed and identified in the signal received in Block 264. Therefore, the algorithm may be applied to the sensed signal in Block 274. In applying the algorithm in Block 274, the signal may be determined to be a true evoked EMG signal or not a true evoked EMG signal.

The method 250 then makes a determination of whether the received sensor signal is a true evoked EMG signal in Block 280. The determination is made by the application of the algorithm, including one or more of the various attributes, including those noted above. Thus, the signal may be determined to be or not be a true or positive evoked EMG signal. If the signal is determined not to be a true evoked EMG signal, a NO path 284 is followed. The NO path 284 may follow a main path to the display 294, as discussed herein. This will allow an output to the user 30 that the signal is not a true evoked EMG signal. The output to the user 30 may, as discussed herein, therefore may be regarding the signal being a true or not a true evoked EMG. The user 30, therefore, may understand the signal to be confirmed as a true evoked EMG or not true evoked EMG. The output, as discussed herein, may differ so the type of the signal determination. In various embodiments, however, the NO path 284 may also/or alternatively go to continue receiving sensor signals in Block 264 and/or receive threshold in block 260. Therefore, the monitoring system 20 may continuously monitor signals received to make a determination whether they are or are not truly an evoked EMG signals when a signal is determined to not be a true evoked EMG.

If a determination is made that the signal is a true or positive evoked EMG signal, a YES path 290 may be followed. The YES path 290 may then allow for an output from the monitoring system 22 to the user 30 that a true evoked EMG signal has been sensed by the monitoring system 20. Once the signal is determined to be a true evoked EMG signal and the YES path 290 is followed, various outputs may be made to the user 30.

In various embodiments, the output signal to the user may be optionally displayed in Block 294. Displaying the signal may be a continuous process where the signal is continuously displayed that is sensed by the sensor 40. The continuous display of the signals in Block 294 may, therefore, include display of true evoked EMG signals and other signals, including possible false positive evoked EMG signals and noise signals. Nevertheless, the continuous display in Block 294 may allow the user 30 understand the signal being continuously received at the monitoring system 20.

Further additional or alternative outputs may be made to the user in Block 300. The monitoring system 20 may determine that the output is made when it is determined that the YES path 290 is followed once the true evoked EMG signal is determined. The outputs may be any appropriate output and may include one or more outputs. The outputs may be a display 304, an audible or sound output 308, and/or a haptic output 312. The display output 304 may include a visual display that a true evoked EMG signal is determined in Block 280. For example, display 22 may flash a color, provide an indica that the signal is a true evoked EMG signal, or other appropriate display. The sound 308 may include an audible sound that may be heard by the user 30 and include one or more tones or the like. The haptic output 312 may include a vibration of the stimulation instrument 50, vibration of an instrument used by the user 30 to perform the procedure on the subject 34 or other appropriate haptic output. Nevertheless, the monitoring system 20 may automatically provide output to the user 30 when a true or non-true evoked EMG signal is determined in Block 280. Also, as noted above, the output may differ based on the determination of the signal as a true evoked EMG or not a true evoked EMG. For example, the display may be green for true evoked EMG and yellow for not true evoked EMG. The haptic feedback may include short vibration bursts for true evoked EMG and long vibration bursts for not true evoked EMG.

After the output to the user 30, a determination of whether the procedure is complete may be made in Block 320. If the procedure is not complete, a NO path 324 may be followed receive threshold in block 260. In various embodiments, the NO path 324 may also or alternatively go to continue receiving sensor signals in Block 264. Thus again, the monitoring system 20 may continuously monitor signals at the sensors 40 for an analysis with a monitoring system 20. However, if the procedure is complete, a YES path 328 may be followed to an END block 330. The procedure completes at 330 may include finalizing a procedure, such as closing an incision and/or other appropriate procedures such as completing recording with the monitoring system 20, analyzing or completing an analysis of receipt of signals by the monitoring system 20, or the like. Further, the user 30 may disengage the monitoring system 20 such that the determination for the signals is not made.

In the method 250, the application of the algorithm in Block 274 and determining whether the sensor signal is a true or positive evoked EMG in Block 280 may be made by the analysis of the received signal with one or more of the attributes, as noted above. As Illustrated in FIG. 3, one or more of the attributes are identified and defined. Accordingly, an algorithm may evaluate the signal to determine whether one or more of the attributes is present in the signal to make a determination of whether the signal is a true evoked EMG signal.

In various embodiments, for example, the algorithm may identify that the signal has at least one of the attributes to make a determination that the received sensor signal is a true evoked EMG signal in Block 280. In various embodiments, only a single one of the attributes may be used to identify the received signal as the true evoked EMG signal. As noted above, more than one of the attributes (e.g., two, three, four, five, or any appropriate number of attributes) may be included in the algorithm. If more than one attribute is used in the algorithm, one or more may be weighted in the algorithm. The weights, if used, may be determined by an appropriate analysis, and either be fixed or determined dynamically. Therefore, two or more of the attributes may be identified in the received signal before a determination is made that the sensor signal is a true evoked EMG.

Additionally, the algorithm may include all of the attributes, including those listed in FIG. 3. Therefore, identification of all of the attributes may be made in the received signal before a determination is made that the signal is a true evoked EMG signal in Block 280.

Thus, the algorithm that is applied in Block 274 may retrieve the attributes to be found or determined in the received signal, such as those illustrated in FIG. 3, to determine whether they are present in the received signal to make the determination that the signal was a true evoked EMG signal in Block 280. When the algorithm determines that the signal is a true evoked signal, one or more of the attributes may be determined to be present in the received signal. The determination that the attribute is present may be based upon one or more of the measurements, as noted above, and/or calculations and/or comparisons based thereon.

In addition, as noted above, various machine learning processes may be used to determine that the attributes are present in the received signal to make the determination that the sensor signal is a true evoked EMG in Block 280. The various machine learning processes can include those discussed above. The machine learning systems may be trained with the various attributes that are identified or labeled in various training signals to train the machine learning system. The machine learning system may then identify whether or not the attributes are present in the received signal to make the determination of whether a sensor signal is a true evoked EMG signal in Block 280.

Regardless, the algorithm may be applied in Block 274 to make the determination of whether a signal is a true EMG signal in Block 280. The application or use of the algorithm may be used to determine the presence of one or more of the attributes, including those listed in FIG. 3, to make the determination that the sensed signal is a true evoked EMG signal. Therefore, the algorithm may make the determination that the signal is not the true evoked EMG signal when the selected attributes are not present in the received signal. This allows the algorithm to determine that the sensed signal is either a true evoked EMG signal or not a true evoked EMG signal according to the method 250, as illustrated in FIG. 4.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a special purpose computer created by configuring a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C #, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include physical connections, such as wired connections. Communications may also or alternatively include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor or module or ‘controller’ may be replaced with the term ‘circuit.’ The term ‘module’ may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system operable to determine a signal as a true or non-true evoked electromyography (EMG), the system comprising:
a processor operable to execute instructions to:
receive a signal from a sensor on a subject;
recall an analysis program;
execute the analysis program to analyze the received signal;
determine the received signal as either a true evoked EMG signal or not true evoked EMG signal based on executing the analysis program; and
output to a user the determination.

2. The system of claim 1, further comprising:
a memory system having stored thereon the analysis program.

3. The system of claim 1, wherein the processor outputs to the user only the determination of the true evoked EMG signal.

4. The system of claim 1, wherein the processor outputs to the user both the determination of the true evoked EMG signal and the not true evoked EMG signal.

5. The system of claim 1, further comprising:
at least one of an audible output system, a visual output system, a haptic output system, or combinations thereof as the output to the user;
wherein the output is made by at least one of the audible output system, the visual output system, the haptic output system, or combinations thereof.

6. The system of claim 1, wherein the analysis program includes instructions to determine the presence of at least one attribute of the signal that is indicative of the true evoked EMG.

7. The system of claim 6, wherein the analysis program includes instructions to identify the presence of at least a waveform of the received signal as the at least one attribute of the signal that is indicative of the true evoked EMG.

8. The system of claim 6, wherein the analysis program determines the presence of a plurality of attributes of the signal that is indicative of the true evoked EMG.

9. The system of claim 8, wherein the analysis program includes instructions to identify the presence of at least one attribute of the signal that is indicative of the true evoked EMG.

10. The system of 6, wherein the at least one attribute includes at least one of an Is_Event, a Peak-to-Peak value, an Onset_Latency, a Peak_Latency, a Waveform, or combinations thereof.

11. The system of claim 5, wherein the at least one attribute includes a Waveform.

12. The system of claim 6, wherein the analysis program includes instructions using a surgical procedure type to determine the presence of at least one attribute of the signal that excludes not true evoked EMG or indicative of the true evoked EMG.

13. A method of determining a signal that is a true or not true evoked electromyography (EMG), the method comprising:
receiving a signal from a sensor on a subject;
recalling an analysis program;
executing the program to analyze the received signal;
determining the received signal as either a true evoked EMG signal or not true evoked EMG; and
outputting to a user the determination.

14. The method of claim 13, wherein outputting to the user the determination includes outputting only the determination of the true evoked EMG signal.

15. The method of claim 14, wherein the output includes at least one of an audible output, a visual output, a haptic output, or combinations thereof.

16. The method of claim 13, wherein the analysis program includes instructions to determine the presence of at least one attribute of the signal that is indicative of the true evoked EMG.

17. The method of claim 16, wherein the analysis program includes instructions to identify the presence of at least one attribute of the signal that is indicative of the true evoked EMG.

18. The method of claim 16, wherein the analysis program determines the presence of a plurality of attributes of the signal that is indicative of the true evoked EMG.

19. The method of claim 18, wherein the analysis program includes instructions to identify the presence of at least one attribute of the signal that is indicative of the true evoked EMG.

20. The method of claim 16, wherein the at least one attribute includes at least one of Is_Event, Peak-to-Peak value, an Onset_Latency, a Peak_Latency, a Waveform, or combinations thereof.

21. The method of claim 16, wherein the at least one attribute includes at least a Waveform.

22. The method of claim 13, wherein the analysis program is a trained machine learning program.
